SEQUENCE LISTING (1 sequence)

SEQ ID NO: 1 - protein, 275 aa, Bacillus subtilis [sequence omitted]

(19) United States
(12) Patent Application Publication
(10) Pub. No.: US 2003/0170846 A1
(43) Pub. Date: Sep. 11, 2003

(54) Enzyme variants having one or more D-amino acid substitutions

(76) Inventor: David John Weisgerber, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 09/824,607

(22) Filed: Apr. 2, 2001

Related U.S. Application Data
(60) Provisional application No. 60/199,415, filed on Apr. 24, 2000.

(51) Int. Cl. C12N 9/02; C12N 9/10; C12N 9/14; C12N 9/24; C12N 9/48

(52) U.S. Cl. 435/193; 435/195; 435/189; 435/200; 435/212

(57) ABSTRACT

The present invention relates to enzyme variants having decreased immunogenicity relative to their corresponding wild-type enzymes. More particularly, the present invention relates to enzyme variants having a modified amino acid sequence of a wild-type amino acid sequence, wherein the modified amino acid sequence comprises a substitution of one or more amino acid positions with at least one D-amino acid. The invention further relates to mutant genes encoding such enzyme variants and cleaning and personal care compositions comprising such enzyme variants.

### CROSS REFERENCE TO RELATED APPLICATION

This application claims the benefit of U.S. Provisional Application No. 60/199,415, filed Apr. 24, 2000.

### FIELD OF THE INVENTION

The present invention relates to enzyme variants which have decreased immunogenicity relative to their corresponding wild-type enzymes, as well as compositions comprising the variants, DNA sequences encoding the variants, and methods of using the variants.

### BACKGROUND OF THE INVENTION

Enzymes make up the largest class of naturally occurring proteins and are responsible for the catalysis of various reactions. For example, one class of enzyme includes proteases which catalyze the hydrolysis of other proteins. This ability to hydrolyze proteins has been exploited by incorporating naturally occurring and protein-engineered proteases into cleaning compositions, particularly those relevant to laundry applications. Other enzymes, e.g., amylases and lipases, are also useful for incorporation into various cleaning compositions for many purposes, including the hydrolysis of starch and lipids.

In the cleaning arts, the mostly widely utilized of these proteases are the serine proteases. Most of these serine proteases are produced by bacterial organisms while some are produced by other organisms, such as fungi. See Siezen, Roland J. et al., “Homology Modelling and Protein Engineering Strategy of Subtilases, the Family of Subtilisin-Like Serine Proteases”, Protein Engineering, Vol. 4, No. 7, pp. 719-737 (1991).

A similar characteristic of all these enzymes relates to the efficacy of the wild-type enzymes in their natural environment relative to the unnatural cleaning composition environment. Typically, the efficacies in the natural environment do not translate to the unnatural environment, rendering the enzyme less useful. For example, protease characteristics such as, for example, thermal stability, pH stability, oxidative stability and substrate specificity are not necessarily optimized for utilization outside the natural environment of the enzyme.

Several approaches have been employed to alter the wild-type amino acid sequence of enzymes with the goal of increasing the efficacy of the enzyme in the unnatural wash environment. These approaches include the genetic redesign of enzymes to enhance, for example, thermal stability and to improve oxidation stability under quite diverse conditions.

However, because such genetically engineered enzymes are foreign to mammals, they are potential antigens. As antigens, these enzymes cause immunological and/or allergenic responses (herein collectively described as allergenic or allergic responses for simplicity) in mammals. In fact, sensitization to enzymes has been observed in environments wherein humans are regularly exposed to the enzymes. These environments include manufacturing facilities, where employees are exposed to the enzymes through such vehicles as uncontrolled dust or aerosolization. Aerosolization can result by the introduction of the enzyme into the lung, which is the route of enzyme exposure which causes the most dangerous response. Enzyme sensitization can also occur in the marketplace, where consumers' repeated use of products containing enzymes may cause an allergic reaction.

Furthermore, while genetic engineering has been prominent in the continuing search for more highly effective enzymes for use in laundry applications, genetically engineered enzymes have been minimally utilized in personal care compositions and light duty detergents. A primary reason for the absence of engineered enzymes in products such as, for example, cosmetics, hand/body lotions and creams, soaps, gels, body washes, and shampoos, is due to the aforementioned problem of human sensitization leading to undesirable allergic responses. It would therefore be highly advantageous to provide a personal care composition which provides the cleansing properties of engineered enzymes with minimized provocation of allergic responses.

One approach toward alleviating the allergenic activity of an enzyme is through the redesign of one or more epitopes of the enzyme. Epitopes are those amino acid regions of an antigen which evoke an immunological response through the binding of antibodies or the presentation of processed antigens to T cells via a major histocompatibility complex protein (MHC). Changes in the epitopes can affect their efficiency as an antigen. See Walsh, B. J. and M. E. H. Howden, “A Method for the Detection of IgE Binding Sequences of Allergens Based on a Modification of Epitope Mapping”, Journal of Immunological Methods, Vol. 121, pp. 275-280 (1989).

It has been discovered that substitution of one or more amino acid residues in the wild-type enzyme with one or more Doppelganger-amino acids (herein referred to as “D-amino acids” for simplicity), particularly wherein the substitution is made in an epitope region, provides an enzyme variant which surprisingly exhibits decreased allergenic and/or immune response while also retaining enzyme efficacy. For reference, amino acids exist in both the Levorotary (“L-amino acids”) and Doppelganger (“D-amino acids”) conformations. L-amino acids are naturally occurring in nature and most biological systems. Additionally, naturally occurring polypeptides are comprised of L-amino acids, and may therefore be referred to as L-polypeptides. D-amino acids are the “mirror images” of their L-amino acid counterparts. Although D-polypeptides (polypeptides fully comprised of D-amino acids) are not naturally occurring, these polypeptides may be synthetically manufactured to form a three-dimensional protein structure.

The present inventors have discovered that inclusion of at least one D-amino acid, preferably in an epitope region of an enzyme, renders that enzyme less allergenic relative to the wild-type enzyme. Without intending to be limited by theory, it is believed that biological systems will not recognize the epitope region containing the D-amino acid as a true epitope region. Thus, the normally occurring allergenic response does not occur. However, excitingly, biological function and enzyme efficacy is maintained. Accordingly, enzymes eliciting decreased allergenic response and maintained enzyme efficacy are provided herein.

Thus, the present invention is directed to enzyme variants having a modified amino acid sequence of a wild-type amino acid sequence, comprising a substitution by a substituting D-amino acid at one or more amino acid positions. Accordingly, the present enzymes are suitable for use in several types of compositions including, but not limited to, laundry, dish, hard surface, skin care, hair care, beauty care, oral, and contact lens compositions.

### SUMMARY OF THE INVENTION

The present invention relates to enzyme variants having decreased immunogenicity relative to their corresponding wild-type enzymes. More particularly, the present invention relates to enzyme variants having a modified amino acid sequence of a wild-type amino acid sequence, wherein the enzyme variant comprises a substitution by a substituting D-amino acid at one or more amino acid positions. At least one amino acid of the enzyme variant is an L-amino acid. The invention further relates to mutant genes encoding such variants and cleaning and personal care compositions comprising such variants.

### DETAILED DESCRIPTION OF THE INVENTION

The essential components of the present invention are described herein. Also included are non-limiting descriptions of various optional and preferred components useful in embodiments of the present invention.

The present invention can comprise, consist of, or consist essentially of any of the required or optional components and/or limitations described herein.

All percentages and ratios are calculated by weight unless otherwise indicated. All percentages are calculated based on the total composition unless otherwise indicated.

All component or composition levels are in reference to the active level of that component or composition, and are exclusive of impurities, for example, residual solvents or by-products, which may be present in commercially available sources.

All documents referred to herein, including all patents, patent applications, and publications, are hereby incorporated by reference in their entirety.

Referred to herein are trade names for materials including, but not limited to, proteins, and more particularly enzymes. The inventors herein do not intend to be limited by materials under a certain trade name. Equivalent materials (e.g., those obtained from a different source under a different name or catalog (reference) number) to those referenced by trade name may be substituted and utilized in the compositions herein.

As used herein, abbreviations will be used to describe amino acids, including Levoratory amino acids (“L-amino acids”) and Doppelganger amino acids (“D-amino acids”). Table 1 provides a list of abbreviations used herein:

TABLE 1

| | Three-letter | One-letter | Three-letter | One-letter |
|---|---|---|---|---|
| | Abbreviation | Abbreviation | Abbreviation | Abbreviation |
| | for L-amino | for L-amino | for D-amino | for D-amino |
| Amino Acid | acid | acid | acid | acid |
| Alanine | Ala | A | D-Ala | D-A |
| Arginine | Arg | R | D-Arg | D-R |
| Asparagine | Asn | N | D-Asn | D-N |
| Aspartic Acid | Asp | D | D-Asp | D-D |
| Cysteine | Cys | C | D-Cys | D-C |
| Glutamine | Gln | Q | D-Gln | D-Q |
| Glutamic Acid | Glu | E | D-Glu | D-E |
| Glycine | Gly | G | D-Gly | D-G |
| Histidine | His | H | D-His | D-H |
| Isoleucine | Ile | I | D-Ile | D-I |
| Leucine | Leu | L | D-Leu | D-L |
| Lysine | Lys | K | D-Lys | D-K |
| Methionine | Met | M | D-Met | D-M |
| Phenylalanine | Phe | F | D-Phe | D-F |
| Proline | Pro | P | D-Pro | D-P |
| Serine | Ser | S | D-Ser | D-S |
| Threonine | Thr | T | D-Thr | D-T |
| Tryptophan | Trp | W | D-Trp | D-W |
| Tyrosine | Tyr | Y | D-Tyr | D-Y |
| Valine | Val | V | D-Val | D-V |

As used herein, a variant may be designated by referring to the substituted amino acid positions which characterize the variant. Substitutions are herein indicated by providing the wild-type amino acid residue, followed by the position number, followed by the substituted amino acid residue to be substituted. Wherein the substituted amino acid residue may be any D-amino acid allowed at that particular position, the symbol “D−*” is provided. Multiple substitutions comprising a variant are separated by the symbol “+”. To illustrate, a substitution of D-valine for glycine at position 70 is designated either Gly70D-Val or G70D-V. An example of a variant having a substitution with a D-amino acid at both positions 70 and 72 may be designated as Gly70D-Val+Val72D-Ala or G70D-V+V72D-A. An example of a variant wherein the substitution is with any D-amino acid at position 76 is designated as N76D−*.

### Definitions

As used herein, the term “mutation” refers to alterations in gene sequences and amino acid sequences produced by those gene sequences. Mutations may be deletions, substitutions, or additions of amino acid residues to the wild-type protein sequence.

As used herein, the term “wild-type” refers to an enzyme produced by unmutated organisms.

As used herein, the term “variant” means an enzyme having an amino acid sequence which differs from that of the corresponding wild-type enzyme.

### Enzyme Variants of the Present Invention

The present inventors have identified certain enzyme variants which exhibit a decreased allergenic response relative to the corresponding wild-type enzyme. It has been discovered that substitution of one or more amino acid residues in the wild-type enzyme with one or more Doppelganger-amino acids (herein referred to as “D-amino acids” for simplicity) provides an enzyme variant which surprisingly exhibits a decreased allergenic response while also retaining enzyme efficacy, particularly in articifical cleaning and personal cleansing environments. Thus, provided herein are enymze variants containing at least one D-amino acid, compositions comprising the variants, DNA sequences coding for the variants, and methods of using the variants, particularly in the cleaning and personal care arts.

For reference, amino acids exist in both the Levorotary (“L-amino acids”) and Doppelganger (“D-amino acids”) conformations. L-amino acids are naturally occurring in nature and most biological systems. Additionally, naturally occurring polypeptides are comprised of L-amino acids, and may therefore be referred to as L-polypeptides. D-amino acids are the “mirror images” of their L-amino acid counterparts. Although D-polypeptides (polypeptides fully comprised of D-amino acids) are not naturally occurring, these polypeptides may be synthetically manufactured to form a three-dimensional protein structure.

The present inventors have discovered that inclusion of at least one D-amino acid, preferably in an epitope region of an enzyme, renders that enzyme less allergenic relative to the corresponding wild-type enzyme. Without intending to be limited by theory, it is believed that biological systems will not recognize the epitope region containing the D-amino acid as a true epitope region. Thus, the normally occurring allergenic response does not occur. However, excitingly, biological function and enzyme efficacy is maintained. Accordingly, enzymes eliciting decreased allergenic response and maintained enzyme efficacy are provided herein. The present enzymes are suitable for use in several types of compositions including, but not limited to, laundry, dish, hard surface, skin care, hair care, beauty care, oral, and contact lens compositions.

Thus, the present invention is directed to enzyme variants having a modified amino acid sequence of a wild-type amino acid sequence, comprising a substitution by a substituting D-amino acid at one or more amino acid positions. Since enzymes fully comprised of D-amino acids should be avoided, at least one amino acid of the variant is an L-amino acid. Preferably, at least one of the substitutions occurs in an epitope region of the wild-type amino acid sequence. The epitope regions include B-cell epitope regions and T-cell epitope regions, however, it is preferred to introduce a substitution with a D-amino acid in at least one T-cell epitope region.

Preferably, the enzyme variant herein is a modified wild type enzyme selected from proteases, cellulases, lipases, amylases, peroxidases, microperoxidases, hemicellulases, xylanases, phsopholipases, esterases, cutinases, pectinases, keratinases, reductases, oxidases, phenoloxidases, lipoxygenases, ligninases, pullulanases, tannases, pentosanases, malanases, β-glucanases, arabinosidases, hyaluronidases, chondroitinases, laccases, transferases, isomerases, lyases, synthetases, and fruit-based enzymes. More preferably, the enzyme variant herein is a modified wild type enzyme selected from proteases, cellulases, lipases, amylases, peroxidases, microperoxidases, phsopholipases, esterases, pectinases, keratinases, reductases, oxidases, β-glucanases, transferases, lyases, synthetases, and fruit-based enzymes. Even more preferably, the enzyme variant herein is a modified wild type enzyme selected from proteases, cellulases, lipases, amylases, peroxidases, phsopholipases, esterases, pectinases, reductases, oxidases, β-glucanases, transferases, and fruit-based enzymes. Most preferably, the enzyme variant herein is a modified wild type enzyme is a protease.

As a non-limiting example, the enzyme phospholipase A2, which contains 134 amino acids, contains several T-cell epitope regions. These epitope regions occur within amino acid position numbers 71-92, 101-118, 104-121, and 108-125. See e.g., Specht et al., “The Murine (H-2k) T-Cell Epitopes of Bee Venom Phospholipase A2 Lie Outside the Active Site of the Enzyme, Int. Arch. Allergy Immunol., Vol. 112, pp. 226-230 (1997) and Blaser, “Allergen Dose Dependent Cytokine Production Regulates Specific IgE and IgG Antibody Production”, New Horizons in Allergy Immunotherapy, p. 295 (1996). In accordance with the present invention, one or more D-amino acids within any of these epitope regions may be introduced such that the allergenic response is diminished. Any D-amino acid may be utilized, preferably those which are “mirror images” of the naturally occurring L-amino acids as set forth in Table 1 herein.

Preferably, wherein the enzyme is a protease, the variants of the present invention are variants of serine proteases. As used herein, the term “serine protease” means a protease which has at least 50%, and preferably 80%, amino acid sequence identity with the sequences for one or more of a subtilisin-like serine protease. A discussion relating to subtilisin-like serine proteases and their homologies may be found in Siezen et al., “Homology Modelling and Protein Engineering Strategy of Subtilases, the Family of Subtilisin-Like Serine Proteases”, Protein Engineering, Vol. 4, No. 7, pp. 719-737 (1991). Preferred serine proteases for substitution with one or more D-amino acids includes subtilisin BPN′, subtilisin Carlsberg, subtilisin DY, subtilisin 309, proteinase K, and thermitase. More preferred serine proteases for substitution with one or more D-amino acids include subtilisin BPN′ and subtilisin 309. The most preferred serine protease for substitution with one or more D-amino acids is subtilisin BPN′.

It has been discovered that epitope regions exist in serine proteases which correspond to positions 70-84, 103-126, and 217-252 of subtilisin BPN′. The present inventors have further discovered that one or more amino acid substitutions, by a substituting D-amino acid, within one or more of these epitope regions provides variants which evoke a decreased allergenic response relative to the corresponding wild-type serine protease. With respect to position notations of serine proteases, and while the variants of the present invention are not limited to those of subtilisin BPN′, all amino acid numbering is with reference to the amino acid sequence for subtilisin BPN′ which is represented by SEQ ID NO:1. The amino acid sequence for subtilisin BPN′ is further described by Wells, J. A., E. Ferrari, D. J. Henner, D. A. Estell, and E. Y. Chen, Nucleic Acids Research, Vol. 11, 7911-7925 (1983). Other epitope regions have been identified in serine proteases, including those described in Loevborg, U.S. Pat. No. 5,766,898, assigned to Novo Nordisk A/S, issued Jun. 16, 1998.

Thus, a particularly preferred embodiment of the present invention relates to enzyme variants having substitutions in one or more of three epitope regions by one or more substituting D-amino acids, wherein the enzyme variant is a serine protease variant. Any D-amino acid may be utilized, preferably those which are “mirror images” of the naturally occurring L-amino acids as set forth in Table 1 herein. The preferred variants have a modified amino acid sequence of a wild-type amino acid sequence, wherein the wild-type amino acid sequence comprises a first epitope region, a second epitope region, and a third epitope region, wherein the modified amino acid sequence comprises a substitution by a substituting D-amino acid at one or more positions in one or more of the epitope regions wherein:

(i) when a substitution occurs in the first epitope region, the substitution occurs at one or more positions corresponding to positions 70-84 of subtilisin BPN′;

(ii) when a substitution occurs in the second epitope region, the substitution occurs at one or more positions corresponding to positions 103-126 of subtilisin BPN′; and

(iii) when a substitution occurs in the third epitope region, the substitution occurs at one or more positions corresponding to positions 217-252 of subtilisin BPN′.

Thus, the variants of the present invention may be variants of serine proteases having a modified amino acid sequence of a wild-type amino acid sequence comprising a substitution by a substituting D-amino acid, wherein the substitutions are at one or more of positions 70, 71, 72, 73, 74, 75, 76, 77, 78, 79, 80, 81, 82, 83, and 84 (70-84), 103, 104, 105, 106, 107, 108, 109, 110, 111, 112, 113, 114, 115, 116, 117, 118, 119, 120, 121, 122, 123, 124, 125, and 126 (103-126), and 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, and 252 (217-252) corresponding to subtilisin BPN′. More preferably, the modified amino acid sequence comprises a substitution of two or more of these positions with two or more D-amino acids, even more preferably three of more of these positions with three or more D-amino acids. Substitutions at these positions are made by replacing the wild-type amino acid residue with a D-amino acid. Preferably, the amino acid utilized for substitution is the D-conformation of a naturally occurring amino acid and thus is made by replacing the wild-type amino acid residue with a D-amino acid residue such as one given in Table 1.

More specifically, the variants have a modified amino acid sequence of a wild-type amino acid sequence comprising a substitution by a substituting D-amino acid at one or more positions corresponding to positions 70-84 of subtilisin BPN′ wherein:

(a) when a substitution occurs at position 70, the substituting D-amino acid is selected from the group consisting of D-Ala, D-Arg, D-Asn, D-Asp, D-Cys, D-Gln, D-Glu, D-His, D-Ile, D-Leu, D-Lys, D-Met, D-Phe, D-Pro, D-Ser, D-Thr, D-Trp, D-Tyr, and D-Val;

(b) when a substitution occurs at position 71, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Trp, D-Tyr, and D-Val; more preferably D-Ala, D-Arg, D-Asn, D-Cys, D-Gly, D-Phe, D-His, D-Ile, D-Lys, D-Leu, D-Met, D-Pro, D-Gln, D-Ser, D-Trp, D-Tyr, and D-Val;

(c) when a substitution occurs at position 72, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, and D-Tyr;

(d) when a substitution occurs at position 73, the substituting D-amino acid is selected from the group consisting of D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val; more preferably D-Arg, D-Asn, D-Asp, D-Cys, D-Glu, D-Gly, D-Phe, D-His, D-Ile, D-Lys, D-Met, D-Pro, D-Ser, D-Thr, D-Trp, D-Tyr, and D-Val;

(e) when a substitution occurs at position 74, the substituting amino acid is selected from the group consisting of D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val; more preferably D-Arg, D-Asn, D-Asp, D-Cys, D-Glu, D-Gly, D-Phe, D-His, D-Ile, D-Lys, D-Leu, D-Met, D-Gln, D-Ser, D-Thr, D-Trp, D-Tyr, and D-Val;

(f) when a substitution occurs at position 75, the substituting amino acid is selected from the group consisting of D-Ala, D-Arg, D-Asn, D-Asp, D-Cys, D-Gln, D-Glu, D-Gly, D-His, D-Ile, D-Lys, D-Met, D-Phe, D-Pro, D-Ser, D-Thr, D-Trp, D-Tyr, and D-Val; more preferably D-Ala, D-Arg, D-Asn, D-Asp, D-Cys, D-Glu, D-Gly, D-Phe, D-His, D-Ile, D-Lys, D-Met, D-Gln, D-Ser, D-Thr, D-Trp, D-Tyr, and D-Val;

(g) when a substitution occurs at position 76, the substituting amino acid is selected from the group consisting of D-Ala, D-Arg, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val; more preferably, D-Ala, D-Arg, D-Cys, D-Ile, D-Leu, D-Met, D-Gln, D-Ser, D-Thr, D-Trp, D-Tyr, and D-Val;

(h) when a substitution occurs at position 77, the substituting amino acid is selected from the group consisting of D-Ala, D-Arg, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val; more preferably D-Ala, D-Arg, D-Cys, D-Glu, D-Gly, D-Phe, D-His, D-Ile, D-Lys, D-Leu, D-Met, D-Pro, D-Gln, D-Ser, D-Thr, D-Trp, D-Tyr, and D-Val;

(i) when a substitution occurs at position 78, the substituting amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Thr, D-Trp, D-Tyr, and D-Val; more preferably D-Ala, D-Arg, D-Asn, D-Cys, D-Glu, D-Gly, D-Phe, D-His, D-Ile, D-Lys, D-Leu, D-Met, D-Pro, D-Gln, D-Thr, D-Trp, D-Tyr, and D-Val;

(j) when a substitution occurs at position 79, the substituting amino acid is selected from the group consisting of D-Ala, D-Arg, D-Asn, D-Asp, D-Cys, D-Gln, D-Glu, D-Gly, D-His, D-Leu, D-Lys, D-Met, D-Phe, D-Pro, D-Ser, D-Thr, D-Trp, D-Tyr, and D-Val; more preferably D-Ala, D-Arg, D-Asn, D-Cys, D-Gly, D-Phe, D-His, D-Lys, D-Leu, D-Met, D-Pro, D-Gln, D-Ser, D-Thr, D-Trp, D-Tyr, and D-Val;

(k) when a substitution occurs at position 80, the substituting amino acid is selected from the group consisting of D-Ala, D-Arg, D-Asn, D-Asp, D-Cys, D-Gln, D-Glu, D-Gly, D-His, D-Ile, D-Lys, D-Met, D-Phe, D-Pro, D-Ser, D-Thr, D-Trp, D-Tyr, and D-Val;

(l) when a substitution occurs at position 81, the substituting amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, and D-Tyr;

(m) when a substitution occurs at position 82, the substituting amino acid is selected from the group consisting of D-Ala, D-Arg, D-Asn, D-Asp, D-Cys, D-Gln, D-Glu, D-Gly, D-His, D-Ile, D-Lys, D-Met, D-Phe, D-Pro, D-Ser, D-Thr, D-Trp, D-Tyr, and D-Val;

(n) when a substitution occurs at position 83, the substituting amino acid is selected from the group consisting of D-Ala, D-Arg, D-Asn, D-Asp, D-Cys, D-Gln, D-Glu, D-His, D-Ile, D-Leu, D-Lys, D-Met, D-Phe, D-Pro, D-Ser, D-Thr, D-Trp, D-Tyr, and D-Val; and

(o) when a substitution occurs at position 84, the substituting amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, and D-Tyr.

Even more preferably, the variants of the present invention comprise a substitution by a substituting D-amino acid of one or more of positions 73, 74, 75, 76, 77, 78, 79, 80, 81, 82, 83 (73-83) corresponding to subtilisin BPN′, even more preferably one or more of 75, 76, 77, 78, 79, 80, 81, 82 (75-82) corresponding to subtilisin BPN′.

The variants may have a modified amino acid sequence of a wild-type amino acid sequence comprising a substitution by a substituting D-amino acid at one or more positions corresponding to positions 103-126 of subtilisin BPN′ wherein:

(a) when a substitution occurs at position 103, the substituting D-amino acid is selected from the group consisting of D-Ala, D-Arg, D-Asn, D-Asp, D-Cys, D-Glu, D-Gly, D-His, D-Ile, D-Leu, D-Lys, D-Met, D-Phe, D-Pro, D-Ser, D-Thr, D-Trp, D-Tyr, and D-Val;

(b) when a substitution occurs at position 104, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, and D-Val;

(c) when a substitution occurs at position 105, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Thr, D-Trp, D-Tyr, and D-Val;

(d) when a substitution occurs at position 106, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Tyr, and D-Val;

(e) when a substitution occurs at position 107, the substituting D-amino acid is selected from the group consisting of D-Ala, D-Arg, D-Asn, D-Asp, D-Cys, D-Gln, D-Glu, D-Gly, D-His, D-Leu, D-Lys, D-Met, D-Phe, D-Pro, D-Ser, D-Thr, D-Trp, D-Tyr, and D-Val;

(f) when a substitution occurs at position 108, the substituting D-amino acid is selected from the group consisting of D-Ala, D-Arg, D-Asn, D-Asp, D-Cys, D-Gln, D-Glu, D-Gly, D-His, D-Leu, D-Lys, D-Met, D-Phe, D-Pro, D-Ser, D-Thr, D-Trp, D-Tyr, and D-Val;

(g) when a substitution occurs at position 109, the substituting D-amino acid is selected from the group consisting of D-Ala, D-Arg, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(h) when a substitution occurs at position 110, the substituting D-amino acid is selected from the group consisting of D-Ala, D-Arg, D-Asn, D-Asp, D-Cys, D-Gln, D-Glu, D-His, D-Ile, D-Leu, D-Lys, D-Met, D-Phe, D-Pro, D-Ser, D-Thr, D-Trp, D-Tyr, and D-Val;

(i) when a substitution occurs at position 111, the substituting D-amino acid is selected from the group consisting of D-Ala, D-Arg, D-Asn, D-Asp, D-Cys, D-Gln, D-Glu, D-Gly, D-His, D-Leu, D-Lys, D-Met, D-Phe, D-Pro, D-Ser, D-Thr, D-Trp, D-Tyr, and D-Val;

(j) when a substitution occurs at position 112, the substituting D-amino acid is selected from the group consisting of D-Ala, D-Arg, D-Asn, D-Asp, D-Cys, D-Gln, D-Glu, D-His, D-Ile, D-Leu, D-Lys, D-Met, D-Phe, D-Pro, D-Ser, D-Thr, D-Trp, D-Tyr, and D-Val;

(k) when a substitution occurs at position 113, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Tyr, and D-Val;

(l) when a substitution occurs at position 114, the substituting D-amino acid is selected from the group consisting of D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(m) when a substitution occurs at position 115, the substituting D-amino acid is selected from the group consisting of D-Ala, D-Arg, D-Asn, D-Asp, D-Cys, D-Gln, D-Glu, D-Gly, D-His, D-Leu, D-Lys, D-Met, D-Phe, D-Pro, D-Ser, D-Thr, D-Trp, D-Tyr, and D-Val;

(n) when a substitution occurs at position 116, the substituting D-amino acid is selected from the group consisting of D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(o) when a substitution occurs at position 117, the substituting D-amino acid is selected from the group consisting of D-Ala, D-Arg, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(p) when a substitution occurs at position 118, the substituting D-amino acid is selected from the group consisting of D-Ala, D-Arg, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(q) when a substitution occurs at position 119, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Phe, D-Pro, D-Ser, D-Thr, D-Trp, D-Tyr, and D-Val;

(r) when a substitution occurs at position 120, the substituting D-amino acid is selected from the group consisting of D-Ala, D-Arg, D-Asn, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(s) when a substitution occurs at position 121, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, and D-Tyr;

(t) when a substitution occurs at position 122, the substituting D-amino acid is selected from the group consisting of D-Ala, D-Arg, D-Asn, D-Asp, D-Cys, D-Gln, D-Glu, D-Gly, D-His, D-Leu, D-Lys, D-Met, D-Phe, D-Pro, D-Ser, D-Thr, D-Trp, D-Tyr, and D-Val;

(u) when a substitution occurs at position 123, the substituting D-amino acid is selected from the group consisting of D-Ala, D-Arg, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(v) when a substitution occurs at position 124, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Phe, D-Pro, D-Ser, D-Thr, D-Trp, D-Tyr, and D-Val;

(w) when a substitution occurs at position 125, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Thr, D-Trp, D-Tyr, and D-Val; and

(x) when a substitution occurs at position 126, the substituting D-amino acid is selected from the group consisting of D-Ala, D-Arg, D-Asn, D-Asp, D-Cys, D-Gln, D-Glu, D-Gly, D-His, D-Ile, D-Lys, D-Met, D-Phe, D-Pro, D-Ser, D-Thr, D-Trp, D-Tyr, and D-Val.

The variants may have a modified amino acid sequence of a wild-type amino acid sequence comprising a substitution by a substituting D-amino acid at one or more positions corresponding to positions 217-252 of subtilisin BPN′ wherein:

(a) when a substitution occurs at position 217, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, and D-Val;

(b) when a substitution occurs at position 218, the substituting D-amino acid is selected from the group consisting of D-Ala, D-Arg, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(c) when a substitution occurs at position 219, the substituting D-amino acid is selected from the group consisting of D-Ala, D-Arg, D-Asn, D-Asp, D-Cys, D-Gln, D-Glu, D-His, D-Ile, D-Leu, D-Lys, D-Met, D-Phe, D-Pro, D-Ser, D-Thr, D-Trp, D-Tyr, and D-Val;

(d) when a substitution occurs at position 220, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Trp, D-Tyr, and D-Val;

(e) when a substitution occurs at position 221, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Thr, D-Trp, D-Tyr, and D-Val;

(f) when a substitution occurs at position 222, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Phe, D-Pro, D-Ser, D-Thr, D-Trp, D-Tyr, and D-Val;

(g) when a substitution occurs at position 223, the substituting D-amino acid is selected from the group consisting of D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(h) when a substitution occurs at position 224, the substituting D-amino acid is selected from the group consisting of D-Ala, D-Arg, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(i) when a substitution occurs at position 225, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Ser, D-Thr, D-Trp, D-Tyr, and D-Val;

(j) when a substitution occurs at position 226, the substituting D-amino acid is selected from the group consisting of D-Ala, D-Arg, D-Asn, D-Asp, D-Cys, D-Gln, D-Glu, D-Gly, D-Ile, D-Leu, D-Lys, D-Met, D-Phe, D-Pro, D-Ser, D-Thr, D-Trp, D-Tyr, and D-Val;

(k) when a substitution occurs at position 227, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, and D-Tyr;

(l) when a substitution occurs at position 228, the substituting D-amino acid is selected from the group consisting of D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(m) when a substitution occurs at position 229, the substituting D-amino acid is selected from the group consisting of D-Ala, D-Arg, D-Asn, D-Asp, D-Cys, D-Gln, D-Glu, D-His, D-Ile, D-Leu, D-Lys, D-Met, D-Phe, D-Pro, D-Ser, D-Thr, D-Trp, D-Tyr, and D-Val;

(n) when a substitution occurs at position 230, the substituting D-amino acid is selected from the group consisting of D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(o) when a substitution occurs at position 231, the substituting D-amino acid is selected from the group consisting of D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(p) when a substitution occurs at position 232, the substituting D-amino acid is selected from the group consisting of D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(q) when a substitution occurs at position 233, the substituting D-amino acid is selected from the group consisting of D-Ala, D-Arg, D-Asn, D-Asp, D-Cys, D-Gln, D-Glu, D-Gly, D-His, D-Ile, D-Lys, D-Met, D-Phe, D-Pro, D-Ser, D-Thr, D-Trp, D-Tyr, and D-Val;

(r) when a substitution occurs at position 234, the substituting D-amino acid is selected from the group consisting of D-Ala, D-Arg, D-Asn, D-Asp, D-Cys, D-Gln, D-Glu, D-Gly, D-His, D-Leu, D-Lys, D-Met, D-Phe, D-Pro, D-Ser, D-Thr, D-Trp, D-Tyr, and D-Val;

(s) when a substitution occurs at position 235, the substituting D-amino acid is selected from the group consisting of D-Ala, D-Arg, D-Asn, D-Asp, D-Cys, D-Gln, D-Glu, D-Gly, D-His, D-Ile, D-Lys, D-Met, D-Phe, D-Pro, D-Ser, D-Thr, D-Trp, D-Tyr, and D-Val;

(t) when a substitution occurs at position 236, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Thr, D-Trp, D-Tyr, and D-Val;

(u) when a substitution occurs at position 237, the substituting D-amino acid is selected from the group consisting of D-Ala, D-Arg, D-Asn, D-Asp, D-Cys, D-Gln, D-Glu, D-Gly, D-His, D-Ile, D-Leu, D-Met, D-Phe, D-Pro, D-Ser, D-Thr, D-Trp, D-Tyr, and D-Val;

(v) when a substitution occurs at position 238, the substituting D-amino acid is selected from the group consisting of D-Ala, D-Arg, D-Asn, D-Asp, D-Cys, D-Gln, D-Glu, D-Gly, D-Ile, D-Leu, D-Lys, D-Met, D-Phe, D-Pro, D-Ser, D-Thr, D-Trp, D-Tyr, and D-Val;

(w) when a substitution occurs at position 239, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Ser, D-Thr, D-Trp, D-Tyr, and D-Val;

(x) when a substitution occurs at position 240, the substituting D-amino acid is selected from the group consisting of D-Ala, D-Arg, D-Asn, D-Asp, D-Cys, D-Gln, D-Glu, D-Gly, D-His, D-Ile, D-Leu, D-Met, D-Phe, D-Pro, D-Ser, D-Thr, D-Trp, D-Tyr, and D-Val;

(y) when a substitution occurs at position 241, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Tyr, and D-Val;

(z) when a substitution occurs at position 242, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Trp, D-Tyr, and D-Val;

(aa) when a substitution occurs at position 243, the substituting D-amino acid is selected from the group consisting of D-Ala, D-Arg, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(bb) when a substitution occurs at position 244, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Trp, D-Tyr, and D-Val;

(cc) when a substitution occurs at position 245, the substituting D-amino acid is selected from the group consisting of D-Ala, D-Arg, D-Asn, D-Asp, D-Cys, D-Glu, D-Gly, D-His, D-Ile, D-Leu, D-Lys, D-Met, D-Phe, D-Pro, D-Ser, D-Thr, D-Trp, D-Tyr, and D-Val;

(dd) when a substitution occurs at position 246, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, and D-Tyr;

(ee) when a substitution occurs at position 247, the substituting D-amino acid is selected from the group consisting of D-Ala, D-Asn, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val;

(ff) when a substitution occurs at position 248, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Thr, D-Trp, D-Tyr, and D-Val;

(gg) when a substitution occurs at position 249, the substituting D-amino acid is selected from the group consisting of D-Ala, D-Arg, D-Asn, D-Asp, D-Cys, D-Gln, D-Glu, D-Gly, D-His, D-Ile, D-Leu, D-Lys, D-Met, D-Phe, D-Pro, D-Thr, D-Trp, D-Tyr, and D-Val;

(hh) when a substitution occurs at position 250, the substituting D-amino acid is selected from the group consisting of D-Ala, D-Arg, D-Asn, D-Asp, D-Cys, D-Gln, D-Glu, D-Gly, D-His, D-Ile, D-Lys, D-Met, D-Phe, D-Pro, D-Ser, D-Thr, D-Trp, D-Tyr, and D-Val;

(ii) when a substitution occurs at position 251, the substituting D-amino acid is selected from the group consisting of D-Ala, D-Arg, D-Asn, D-Asp, D-Cys, D-Gln, D-Gly, D-His, D-Ile, D-Leu, D-Lys, D-Met, D-Phe, D-Pro, D-Ser, D-Thr, D-Trp, D-Tyr, and D-Val; and

(jj) when a substitution occurs at position 252, the substituting D-amino acid is selected from the group consisting of D-Ala, D-Arg, D-Asp, D-Cys, D-Gln, D-Glu, D-Gly, D-His, D-Ile, D-Leu, D-Lys, D-Met, D-Phe, D-Pro, D-Ser, D-Thr, D-Trp, D-Tyr, and D-Val.

In a particularly preferred embodiment of the present invention, a D-amino acid substitutes for the wild-type amino acid at one or more of positions 70-84 corresponding to subtilisin BPN′, more preferably positions 73-83 corresponding to subtilisin BPN′, even more preferably positions 70 and 75-82 corresponding to subtilisin BPN′, and most preferably positions 75-82 corresponding to subtilisin BPN′. In a further particularly preferred embodiment of the present invention, a D-amino acid substitutes for the wild-type amino acid at one or more of positions 70-84 corresponding to subtilisin BPN′ (first epitope region) and one or more of positions 103-126 corresponding to subtilisin BPN′ (second epitope region). In another particularly preferred embodiment of the present invention, a D-amino acid substitutes for the wild-type amino acid at one or more of positions 70-84 corresponding to subtilisin BPN′ (first epitope region) and one or more of positions 217-252 corresponding to subtilisin BPN′ (third epitope region). In another preferred embodiment of the present invention, a D-amino acid substitutes for the wild-type amino acid at one or more of positions 70-84 corresponding to subtilisin BPN′ (first epitope region), one or more of positions 103-126 corresponding to subtilisin BPN′ (second epitope region), and one or more of positions 217-252 corresponding to subtilisin BPN′ (third epitope region).

Tables 2-3 below exemplify non-limiting preferred variants of the present invention, wherein serine proteases are utilized for this exemplification. While not specifically illustrated, triple, quadruple, quintuple, sextuple, septuple, octuple, nonuple, and even higher instances of substitutions may be made to form the variant. With respect to these tables, in describing the specific substitutions, the wild-type amino acid residue is given first, the position number (corresponding to subtilisin BPN′) is given second, and the substituting D-amino acid is given third. Tables 2-3 delineate preferred variants having two or three substitutions. To illustrate, the first example of Table 3 reads “VAL 81 D-THR LEU 82 D-PHE”. This example is meant to exemplify the variant Va181D-Thr +Leu82D-Phe.

TABLE 2
Single Substitution Variants

| | | |
|---|---|---|
| GLY | 83 | D-VAL |
| LEU | 82 | D-PHE |
| LEU | 82 | D-HIS |
| LEU | 82 | D-TYR |
| LEU | 82 | D-ALA |
| VAL | 81 | D-THR |
| VAL | 81 | D-ALA |
| GLY | 80 | D-VAL |
| ILE | 79 | D-LEU |
| ILE | 79 | D-MET |
| ILE | 79 | D-THR |
| ILE | 79 | D-VAL |
| ILE | 79 | D-ALA |
| SER | 78 | D-ASN |
| SER | 78 | D-THR |
| SER | 78 | D-ARG |
| SER | 78 | D-GLN |
| SER | 78 | D-HIS |
| SER | 78 | D-LYS |
| SER | 78 | D-TYR |
| SER | 78 | D-ALA |
| ASN | 77 | D-ALA |
| ASN | 76 | D-ALA |
| LEU | 75 | D-ILE |
| LEU | 75 | D-MET |
| LEU | 75 | D-VAL |
| LEU | 75 | D-ALA |
| ALA | 74 | D-VAL |
| ALA | 73 | D-VAL |
| VAL | 72 | D-ALA |
| THR | 71 | D-ALA |
| GLY | 70 | D-VAL |

TABLE 3
Double Substitution Variants

| | | | | | |
|---|---|---|---|---|---|
| VAL | 81 | D-THR | LEU | 82 | D-PHE |
| VAL | 81 | D-THR | LEU | 82 | D-HIS |
| VAL | 81 | D-THR | LEU | 82 | D-TYR |
| ILE | 79 | D-LEU | LEU | 82 | D-PHE |
| ILE | 79 | D-LEU | LEU | 82 | D-HIS |
| ILE | 79 | D-LEU | LEU | 82 | D-TYR |
| ILE | 79 | D-MET | LEU | 82 | D-PHE |
| ILE | 79 | D-MET | LEU | 82 | D-HIS |
| ILE | 79 | D-MET | LEU | 82 | D-TYR |
| ILE | 79 | D-THR | LEU | 82 | D-PHE |
| ILE | 79 | D-THR | LEU | 82 | D-HIS |
| ILE | 79 | D-THR | LEU | 82 | D-TYR |
| ILE | 79 | D-VAL | LEU | 82 | D-PHE |
| ILE | 79 | D-VAL | LEU | 82 | D-HIS |
| ILE | 79 | D-VAL | LEU | 82 | D-TYR |
| ILE | 79 | D-LEU | VAL | 81 | D-THR |
| ILE | 79 | D-MET | VAL | 81 | D-THR |
| ILE | 79 | D-THR | VAL | 81 | D-THR |
| ILE | 79 | D-VAL | VAL | 81 | D-THR |
| SER | 78 | D-ASN | LEU | 82 | D-PHE |
| SER | 78 | D-ASN | LEU | 82 | D-HIS |
| SER | 78 | D-ASN | LEU | 82 | D-TYR |
| SER | 78 | D-THR | LEU | 82 | D-PHE |
| SER | 78 | D-THR | LEU | 82 | D-HIS |
| SER | 78 | D-THR | LEU | 82 | D-TYR |
| SER | 78 | D-ARG | LEU | 82 | D-PHE |
| SER | 78 | D-ARG | LEU | 82 | D-HIS |
| SER | 78 | D-ARG | LEU | 82 | D-TYR |
| SER | 78 | D-ASP | LEU | 82 | D-PHE |
| SER | 78 | D-ASP | LEU | 82 | D-HIS |
| SER | 78 | D-ASP | LEU | 82 | D-TYR |
| SER | 78 | D-GLN | LEU | 82 | D-PHE |
| SER | 78 | D-GLN | LEU | 82 | D-HIS |
| SER | 78 | D-GLN | LEU | 82 | D-TYR |
| SER | 78 | D-HIS | LEU | 82 | D-PHE |
| SER | 78 | D-HIS | LEU | 82 | D-HIS |
| SER | 78 | D-HIS | LEU | 82 | D-TYR |
| SER | 78 | D-LYS | LEU | 82 | D-PHE |
| SER | 78 | D-LYS | LEU | 82 | D-HIS |
| SER | 78 | D-LYS | LEU | 82 | D-TYR |
| SER | 78 | D-TYR | LEU | 82 | D-PHE |
| SER | 78 | D-TYR | LEU | 82 | D-HIS |
| SER | 78 | D-TYR | LEU | 82 | D-TYR |
| SER | 78 | D-ASN | VAL | 81 | D-THR |
| SER | 78 | D-THR | VAL | 81 | D-THR |
| SER | 78 | D-ARG | VAL | 81 | D-THR |
| SER | 78 | D-ASP | VAL | 81 | D-THR |
| SER | 78 | D-GLN | VAL | 81 | D-THR |
| SER | 78 | D-HIS | VAL | 81 | D-THR |
| SER | 78 | D-LYS | VAL | 81 | D-THR |
| SER | 78 | D-TYR | VAL | 81 | D-THR |
| SER | 78 | D-ASN | ILE | 79 | D-LEU |
| SER | 78 | D-ASN | ILE | 79 | D-MET |
| SER | 78 | D-ASN | ILE | 79 | D-THR |
| SER | 78 | D-ASN | ILE | 79 | D-VAL |
| SER | 78 | D-THR | ILE | 79 | D-LEU |
| SER | 78 | D-THR | ILE | 79 | D-MET |
| SER | 78 | D-THR | ILE | 79 | D-THR |
| SER | 78 | D-THR | ILE | 79 | D-VAL |
| SER | 78 | D-ARG | ILE | 79 | D-LEU |
| SER | 78 | D-ARG | ILE | 79 | D-MET |
| SER | 78 | D-ARG | ILE | 79 | D-THR |
| SER | 78 | D-ARG | ILE | 79 | D-VAL |
| SER | 78 | D-ASP | ILE | 79 | D-LEU |
| SER | 78 | D-ASP | ILE | 79 | D-MET |
| SER | 78 | D-ASP | ILE | 79 | D-THR |
| SER | 78 | D-ASP | ILE | 79 | D-VAL |
| SER | 78 | D-GLN | ILE | 79 | D-LEU |
| SER | 78 | D-GLN | ILE | 79 | D-MET |
| SER | 78 | D-GLN | ILE | 79 | D-THR |
| SER | 78 | D-GLN | ILE | 79 | D-VAL |
| SER | 78 | D-HIS | ILE | 79 | D-LEU |
| SER | 78 | D-HIS | ILE | 79 | D-MET |
| SER | 78 | D-HIS | ILE | 79 | D-THR |
| SER | 78 | D-HIS | ILE | 79 | D-VAL |
| SER | 78 | D-LYS | ILE | 79 | D-LEU |
| SER | 78 | D-LYS | ILE | 79 | D-MET |
| SER | 78 | D-LYS | ILE | 79 | D-THR |
| SER | 78 | D-LYS | ILE | 79 | D-VAL |
| SER | 78 | D-TYR | ILE | 79 | D-LEU |
| SER | 78 | D-TYR | ILE | 79 | D-MET |
| SER | 78 | D-TYR | ILE | 79 | D-THR |
| SER | 78 | D-TYR | ILE | 79 | D-VAL |
| ASN | 76 | D-HIS | LEU | 82 | D-PHE |
| ASN | 76 | D-HIS | LEU | 82 | D-HIS |
| ASN | 76 | D-HIS | LEU | 82 | D-TYR |
| ASN | 76 | D-HIS | VAL | 81 | D-THR |
| ASN | 76 | D-HIS | ILE | 79 | D-LEU |
| ASN | 76 | D-HIS | ILE | 79 | D-MET |
| ASN | 76 | D-HIS | ILE | 79 | D-THR |
| ASN | 76 | D-HIS | ILE | 79 | D-VAL |
| ASN | 76 | D-HIS | SER | 78 | D-ASN |
| ASN | 76 | D-HIS | SER | 78 | D-THR |
| ASN | 76 | D-HIS | SER | 78 | D-ARG |
| ASN | 76 | D-HIS | SER | 78 | D-ASP |
| ASN | 76 | D-HIS | SER | 78 | D-GLN |
| ASN | 76 | D-HIS | SER | 78 | D-HIS |
| ASN | 76 | D-HIS | SER | 78 | D-LYS |
| ASN | 76 | D-HIS | SER | 78 | D-TYR |
| LEU | 75 | D-ILE | LEU | 82 | D-PHE |
| LEU | 75 | D-ILE | LEU | 82 | D-HIS |
| LEU | 75 | D-ILE | LEU | 82 | D-TYR |
| LEU | 75 | D-MET | LEU | 82 | D-PHE |
| LEU | 75 | D-MET | LEU | 82 | D-HIS |
| LEU | 75 | D-MET | LEU | 82 | D-TYR |
| LEU | 75 | D-VAL | LEU | 82 | D-PHE |
| LEU | 75 | D-VAL | LEU | 82 | D-HIS |
| LEU | 75 | D-VAL | LEU | 82 | D-TYR |
| LEU | 75 | D-ILE | VAL | 81 | D-THR |
| LEU | 75 | D-MET | VAL | 81 | D-THR |
| LEU | 75 | D-VAL | VAL | 81 | D-THR |
| LEU | 75 | D-ILE | ILE | 79 | D-LEU |
| LEU | 75 | D-ILE | ILE | 79 | D-MET |
| LEU | 75 | D-ILE | ILE | 79 | D-THR |
| LEU | 75 | D-ILE | ILE | 79 | D-VAL |
| LEU | 75 | D-MET | ILE | 79 | D-LEU |
| LEU | 75 | D-MET | ILE | 79 | D-MET |
| LEU | 75 | D-MET | ILE | 79 | D-THR |
| LEU | 75 | D-MET | ILE | 79 | D-VAL |
| LEU | 75 | D-VAL | ILE | 79 | D-LEU |
| LEU | 75 | D-VAL | ILE | 79 | D-MET |
| LEU | 75 | D-VAL | ILE | 79 | D-THR |
| LEU | 75 | D-VAL | ILE | 79 | D-VAL |
| LEU | 75 | D-ILE | SER | 78 | D-ASN |
| LEU | 75 | D-ILE | SER | 78 | D-THR |
| LEU | 75 | D-ILE | SER | 78 | D-ARG |
| LEU | 75 | D-ILE | SER | 78 | D-ASP |
| LEU | 75 | D-ILE | SER | 78 | D-GLN |
| LEU | 75 | D-ILE | SER | 78 | D-HIS |
| LEU | 75 | D-ILE | SER | 78 | D-LYS |
| LEU | 75 | D-ILE | SER | 78 | D-TYR |
| LEU | 75 | D-MET | SER | 78 | D-ASN |
| LEU | 75 | D-MET | SER | 78 | D-THR |
| LEU | 75 | D-MET | SER | 78 | D-ARG |
| LEU | 75 | D-MET | SER | 78 | D-ASP |
| LEU | 75 | D-MET | SER | 78 | D-GLN |
| LEU | 75 | D-MET | SER | 78 | D-HIS |
| LEU | 75 | D-MET | SER | 78 | D-LYS |
| LEU | 75 | D-MET | SER | 78 | D-TYR |
| LEU | 75 | D-AL | SER | 78 | D-ASN |
| LEU | 75 | D-VAL | SER | 78 | D-THR |
| LEU | 75 | D-VAL | SER | 78 | D-ARG |
| LEU | 75 | D-VAL | SER | 78 | D-ASP |
| LEU | 75 | D-VAL | SER | 78 | D-GLN |
| LEU | 75 | D-VAL | SER | 78 | D-HIS |
| LEU | 75 | D-VAL | SER | 78 | D-LYS |
| LEU | 75 | D-VAL | SER | 78 | D-TYR |
| LEU | 75 | D-ILE | ASN | 76 | D-HIS |
| LEU | 75 | D-MET | ASN | 76 | D-HIS |
| LEU | 75 | D-VAL | ASN | 76 | D-HIS |

One or more additional substitutions (“stabilizing substitutions”) with one or more L-amino acids or D-amino acids, preferably L-amino acids, wherein the substitution is made within or outside the epitope region of the enzyme may additionally be made. Such stabilizing substitutions may restabilize the enzyme upon substitution of the epitope region with the D-amino acid or enhance the enzymatic activity of the variant. Many such stabilizing substitutions are well known in the art. Non-limiting examples of such stabilizing mutations (in serine proteases, for example) are disclosed in, for example, WO 95/10591, Baeck et al., published Apr. 20, 1995; U.S. Pat. No. 4,914,031, Zukowski et al., issued Apr. 3, 1990; U.S. Pat. No. 5,470,733, Bryan et al., issued Nov. 28, 1995; U.S. Pat. No. 5,567,601, Bryan et al., issued Oct. 22, 1996; WO 89/07642, U.S. Pat. No. 5,707,848, Bryan et al., issued Jan. 13, 1998; Van Eekelen et al., published Aug. 24, 1989; WO 87/04461, Stabinsky et al., published Jul. 30, 1987; U.S. Pat. No. 4,760,025, Estell et al., issued Jul. 26, 1988; WO 92/11348, Branner et al., published Jul. 9, 1992; EP 0,405,901, Casteleijn et al., published Jan. 2, 1991; WO 91/00345, Branner et al., published Jan. 10, 1991; and WO 94/10020, Brode et al., published Mar. 23, 1995.

Preferred stabilizing substitutions for serine proteases include one or more of: 1107V; K213R; Y217L; Y217K; N218S; G169A; M50F; Q19E; P5A; S9A; 131L; E156S; G169A; N212G; S188P; T254A; S3C+Q206C; and Q271E, wherein (as throughout) the position numbering is with respect to subtilisin BPN′. Among these, the more preferred stabilizing mutations include one or more of P5A; S9A; 131L; E156S; G169A; N212G; S188P; T254A; S3C+Q206C; Q271E; Y217L; and Y217K. The most preferred stabilizing mutations for serine proteases include Y217L and Y217K.

### Method of Making

The present variants may be prepared through synthetic contruction of the enzyme containing one or more site-specific D-amino acid substitutions. Such methods are well-known in the art; one such method is set forth below, using Bacillus subtilis as a non-limiting example. Other methods of preparing the present variants will be known to one of ordinary skill, and may be utilized herein.

Standard solid-phase techniques are used to construct segments of subtilisin BPN′ which are approximately 50 amino acids in length. The segements should span the amino sequence of the propeptide leader sequence as well as the sequence of the final processed form of the protease. The amino acids are incorporated into the appropriate peptide segment by substituting the D-amino acid precursor in place of the corresponding L-amino acid. The peptides should have a reactive leaving group on the C-terminal ends (except for the C-terminal segment of the protein) and a removable protecting group on the amino terminal end (except for the N-terminal segment of the protein). The synthesis is similar to that which is set forth in Abrahmsen et al., WO94/18329, assigned to Genentech, Inc. The modified subtilisin is used to sequentially ligate the synthetic peptide segments in the proper order to create the entire protein including the leader peptide. The protein is folded and autocatalytically processed to release the propeptide and to achieve the active protease (see e.g., Strausberg et al., “Catalysis of a Protein Folding Reaction: Thermodynamic and Kinetic Analysis of Subtilisin BPN′ Interactions with Its Propeptide Fragment”, Biochemistry, Vol. 33, pp. 8112-8119 (1993).

### Analytical Methods

The present variants may be tested for enzymatic activity and allergenic response using the following methods, both of which are known to one ordinarily skilled in the art. Alternatively, other methods well-known in the art may be used.

### Enzyme Variant Activity

The enzyme activity of a variant of the present invention may be assayed by methods which are well-known in the art. Two such methods are set forth herein below, particularly wherein the enzyme is a protease:

Skin Flake Activity Method

This method is particularly useful for determining efficacy of enzyme variants utilized in personal care compositions. Using Scotch® #3750G tape, human skin flakes are stripped from the legs of a subject repeatedly until the tape is substantially opaque with flakes. The tape is then cut into 1 inch by 1 inch squares and set aside. In a 10 mm by 35 mm petri dish, 2 mL of 0.75 mg/mL of a control enzyme (for example, subtilisin BPN′) or the variant to be tested is added in 0.01 M KH₂PO₄ pH 5.5 buffer. To this solution 1 mL of 2.5% sodium laurate pH 8.6 solution is added. The solution is gently mixed on a platform shaker. The previously prepared tape square is soaked in the solution (flake side up) for ten minutes continuing gentle mixing. The tape square is then rinsed gently in tap water for fifteen seconds. Stevenel Blue Stain (3 mL, commercially available from Sigma Chemical Co., St. Louis, Mo.) is pipetted into a clean petri dish. The rinsed tape square is placed into the stain for three minutes (flake side up) with gentle mixing. The tape square is removed from the stain and rinsed consecutively in two beakers of 300 mL distilled water, for fifteen seconds per rinse. The tape square is allowed to air-dry. The color intensity between the tape square obtained from the control enzyme and the tape square obtained from the variant is compared visually or by using a chromameter. Relative to the control enzyme tape square, a variant tape square showing less color intensity is indicative of a variant having higher activity.

Dyed Collagen Activity Method

This method is particularly useful for determining efficacy of enzyme variants utilized in cleaning compositions. Combine 50 mL of 0.1 M tris buffer (tris-hydroxymethyl-aminomethane) containing 0.01 M CaCl₂ to give pH 8.6, and 0.5 g azocoll (azo dye impregnated collagen, commercially available from Sigma Chemical Co., St. Louis, Mo.). Incubate this mixture at 25° C. while gently mixing with a platform shaker. Filter 2 mL of the mixture through a 0.2 micron syringe filter and read absorbance of the mixture at 520 nm to zero a spectrophotometer. Add 1 ppm of a control enzyme (for example, subtilisin BPN′) or the variant to be tested to the remaining 48 mL of tris/azocoll mixture. Filter 2 mL of the control/variant containing solution through a 0.2 micron syringe filter every two minutes for a total of ten minutes. For each filtered sample, read the absorbance immediately at 520 nm. Plot the results against time. The slopes of the control and the test variant are indicative of relative activities of the samples. A higher slope is indicative of a higher activity. The test variant activity (slope) may be expressed as a percent of the control activity (slope).

Mouse Intranasal Test for Immunogenicity

The allergenic potential of the enzymes of the present invention may be determined using a method known in the art or by the Mouse Intranasal Test for Allergenicity presented herein below. This test is similar to the assays described in Robinson et al., “Specific Antibody Responses to Subtilisin Carlsberg (Alcalase) in Mice: Development of an Intranasal Exposure Model”, Fundamental and Applied Toxicology, Vol. 34, pp. 15-24 (1996) and Robinson et al., “Use of the Mouse Intranasal Test (MINT) to Determine the Allergenic Potency of Detergent Enzymes: Comparison to the Guinea Pig Intratracheal (GPIT) Test”, Toxicological Science, Vol. 43, pp. 39-46 (1998), both of which assays may be utilized in place of the test set forth herein below.

Female BDF1 mice (Charles River Laboratories, Portage, Mich.) weighing from about 18 to about 20 grams are utilized in the test. The mice are quarantined one week prior to dosing. The mice are housed in cages with wood chip bedding in rooms controlled for humidity (30-70%), temperature (67-77° F.) and 12 hour light and dark cycles. The mice are fed Purina® mouse chow (Purina Mills, Richmond, Ind.) and water ad libitum.

The enzyme to be tested is dosed to a group of five mice. Prior to dosing, each mouse is anesthetized by an intraperitoneal (i.p.) injection of a mixture of Ketaset (88.8 mg/kg) and Rompun (6.67 mg/kg). The anesthetized animal is held in the palm of the hand, back down, and dosed intranasally with 5 mL enzyme in buffer solution (0.01 M KH₂PO₄, pH 5.5). While each group receives the same dosage, various dosages may be tested. Dosing solutions are gently placed on the outside of each nostril and inhaled by the mouse. Dosing is repeated on days 3, 10, 17, and 24.

Serum samples are collected on day 29. Total and antigen-specific antibodies are measured as described below using ELISA methodolgies.

Measurement of Total IgE

96-well microtiter plates are coated with anti-mouse IgE antibody (Pharmingen). After blocking with BSA in PBS, dilutions of mouse sera are incubated in the wells. After washing, the serum IgE is captured using biotinylated anti-mouse IgE with streptavidin conjugated with horseradish peroxidase. Enzyme development was used with tetramethyl benzidine and hydrogen peroxide quenched with sulfuric acid. Plates are read at 450 nm. OD450 values of appropriate dilutions are compared to a standard curve generated by murine IgE standards to determine concentration of IgE.

Measurement of Antigen-Specific IgE

96-well microtiter plates are coated with anti-mouse IgE antibody (Pharmingen). After blocking with BSA in PBS, dilutions of mouse sera are incubated in the wells. After washing, the serum enzyme-specific IgE is captured using monobiotinylated antigen with streptavidin conjugated with horseradish peroxidase. Enzyme development is used with tetramethyl benzidine and hydrogen peroxide quenched with sulfuric acid. Plates are read at 450 nm. OD450 values of appropriate dilutions are compared to each other to determine relative amounts of antigen-specific IgE.

### T-Cell Proliferation Assay

The allergenic potential of the variants of the present invention may be determined using a T-cell proliferation assay such as the assay presented hereinbelow. This assay is a variation of the assay disclosed in Bungy Poor Fard et al., “T Cell Epitopes of the Major Fraction of Rye Grass Lolium perenne (Lol p I) Defined Using Overlapping Peptides in vitro and in vivo”, Clinical Experimental Immunology, Vol. 94, pp. 111-116 (1993), using subtilisin BPN′ for exemplification purposes.

The blood of subjects allergic to subtilisin BPN′ (prick test positive) and control subjects (prick test negative) are used in this assay. Blood (˜60 mL) from each subject is collected and mononuclear cells are harvested using ficoll-hypaque (which may be obtained from Pharmacia, Piscataway, N.J.). The cells are washed twice in RPMI 1640 (which may be obtained from Gibco, Grand Island, N.Y.) and then resuspended in complete medium RPMI supplemented with 10% human AB-serum, 2 mM L-glutamine, and 25 mg/mL gentamicin (which may be obtained from Gibco). Cells are cultured at a concentration of 2×10⁵ cells/well in 0.2 mL of complete medium in U-bottomed 96-well microtiter plates. The potential antigen to be tested (either inactivated BPN′ as positive control or a variant of the present invention is added at a final concentration up to about 40 mg/mL. Cultures are incubated at 37° C. in 5% CO₂. After five days, 1 mCi/well of methyl-³H-thymidine is added and 18 hours later the cells are harvested. ³H-thymidine incorporation by the cell is assessed as a measure of T-cell proliferation by liquid scintillation counting.

### Compositions of the Present Invention

The variants herein can be used in any application which is suitable for the respective wild-type enzyme. One such example includes cleaning compositions. Because of the desirable reduced allergenicity and/or immunogenicity properties of the present variants, the variants may further be used in applications which have minimally benefitted from the use of enzymes. Examples of such applications include those in which the variant necessarily comes in close contact with human skin, such as with the use of personal care compositions.

### Cleaning Compositions

The variants may be utilized in cleaning compositions including, but not limited to, laundry compositions, hard surface cleansing compositions, light duty cleaning compositions including dish cleansing compositions, and automatic dishwasher detergent compositions.

The cleaning compositions herein comprise an effective amount of one or more variants of the present invention and a cleaning composition carrier.

As used herein, “effective amount of variant”, or the like, refers to the quantity of variant necessary to achieve the proteolytic activity necessary in the specific cleaning composition. Such effective amounts are readily ascertained by one of ordinary skill in the art and is based on many factors, such as the particular variant used, the cleaning application, the specific composition of the cleaning composition, and whether a liquid or dry (e.g., granular, bar) composition is desired, and the like. Preferably, the cleaning compositions comprise from about 0.0001% to about 10%, more preferably from about 0.001% to about 1%, and most preferably from about 0.01% to about 0.1% of one or more variants of the present invention. Several examples of various cleaning compositions wherein the variants may be employed are discussed in further detail below.

In addition to the present variants, the present cleaning compositions further comprise a cleaning composition carrier comprising one or more cleaning composition materials compatible with the variant. The term “cleaning composition material”, as used herein, means any material selected for the particular type of cleaning composition desired and the form of the product (e.g., liquid, granule, bar, spray, stick, paste, gel), which materials are also compatible with the variant used in the composition. The specific selection of cleaning composition materials is readily made by considering the material to be cleaned, the desired form of the composition for the cleaning condition during use. The term “compatible”, as used herein, means the cleaning composition materials do not reduce the proteolytic activity of the variant to such an extent that the variant is not effective as desired during normal use situations. Specific cleaning composition materials are exemplified in detail hereinafter.

The variants of the present invention may be used in a variety of detergent compositions where high sudsing and good cleansing activity is desired. Thus, the variants can be used with various conventional ingredients to provide fully-formulated hard-surface cleaners, dishwashing compositions, fabric laundering compositions, and the like. Such compositions can be in the form of liquids, granules, bars, and the like. Such compositions can be formulated as “concentrated” detergents which contain as much as from about 30% to about 60% by weight of surfactants.

The cleaning compositions herein may optionally, and preferably, contain various surfactants (e.g., anionic, nonionic, or zwitterionic surfactants). Such surfactants are typically present at levels of from about 5% to about 35% of the compositions.

Nonlimiting examples of surfactants useful herein include the conventional C₁₁-C₁₈ alkyl benzene sulfonates and primary and random alkyl sulfates, the C₁₀-C₁₈ secondary (2,3) alkyl sulfates of the formulas CH₃(CH₂)ₓ(CHOSO₃)⁻M⁺)CH₃ and CH₃(CH₂)_{y}(CHOSO₃−M⁺) CH₂CH₃ wherein x and (y+1) are integers of at least about 7, preferably at least about 9, and M is a water-solubilizing cation, especially sodium, the C₁₀-C₁₈ alkyl alkoxy sulfates (especially EO 1-5 ethoxy sulfates), C₁₀-C₁₈ alkyl alkoxy carboxylates (especially the EO 1-5 ethoxycarboxylates), the C₁₀-C₁₈ alkyl polyglycosides, and their corresponding sulfated polyglycosides, C₁₂-C₁₈ a-sulfonated fatty acid esters, C₁₂-C₁₈ alkyl and alkyl phenol alkoxylates (especially ethoxylates and mixed ethoxy/propoxy), C₁₂-C₁₈ betaines and sulfobetaines (“sultaines”), C₁₀-C₁₈ amine oxides, and the like. The alkyl alkoxy sulfates (AES) and alkyl alkoxy carboxylates (AEC) are preferred herein. The use of such surfactants in combination with the amine oxide and/or betaine or sultaine surfactants is also preferred, depending on the desires of the formulator. Other conventional useful surfactants are listed in standard texts. Particularly useful surfactants include the C₁₀-C₁₈ N-methyl glucamides disclosed in U.S. Pat. No. 5,194,639, Connor et al., issued Mar. 16, 1993.

A wide variety of other ingredients useful in detergent cleaning compositions can be included in the compositions herein including, for example, other active ingredients, carriers, hydrotropes, processing aids, dyes or pigments, and solvents for liquid formulations. If an additional increment of sudsing is desired, suds boosters such as the C₁₀-C₁₆ alkolamides can be incorporated into the compositions, typically at about 1% to about 10% levels. The C₁₀-C₁₄ monoethanol and diethanol amides illustrate a typical class of such suds boosters. Use of such suds boosters with high sudsing adjunct surfactants such as the amine oxides, betaines and sultaines noted above is also advantageous. If desired, soluble magnesium salts such as MgCl₂, MgSO₄, and the like, can be added at levels of, typically, from about 0.1% to about 2%, to provide additional sudsing.

The liquid detergent compositions herein may contain water and other solvents as carriers. Low molecular weight primary or secondary alcohols exemplified by methanol, ethanol, propanol, and iso-propanol are suitable. Monohydric alcohols are preferred for solubilizing surfactants, but polyols such as those containing from about 2 to about 6 carbon atoms and from about 2 to about 6 hydroxy groups (e.g., 1,3-propanediol, ethylene glycol, glycerine, and 1,2-propanediol) can also be used. The compositions may contain from about 5% to about 90%, typically from about 10% to about 50% of such carriers.

The detergent compositions herein will preferably be formulated such that during use in aqueous cleaning operations, the wash water will have a pH between about 6.8 and about 11. Finished products are typically formulated at this range. Techniques for controlling pH at recommended usage levels include the use of, for example, buffers, alkalis, and acids. Such techniques are well known to those skilled in the art.

When formulating the hard surface cleaning compositions and fabric cleaning compositions of the present invention, the formulator may wish to employ various builders at levels from about 5% to about 50% by weight. Typical builders include the 1-10 micron zeolites, polycarboxylates such as citrate and oxydisuccinates, layered silicates, phosphates, and the like. Other conventional builders are listed in standard formularies.

Likewise, the formulator may wish to employ various additional enzymes, such as cellulases, lipases, amylases and proteases in such compositions, typically at levels of from about 0.001% to about 1% by weight. Various detersive and fabric care enzymes are well-known in the laundry detergent art.

Various bleaching compounds, such as the percarbonates, perborates and the like, can be used in such compositions, typically at levels from about 1% to about 15% by weight. If desired, such compositions can also contain bleach activators such as tetraacetyl ethylenediamine, nonanoyloxybenzene sulfonate, and the like, which are also known in the art. Usage levels typically range from about 1% to about 10% by weight.

Soil release agents, especially of the anionic oligoester type, chelating agents, especially the aminophosphonates and ethylenediaminedisuccinates, clay soil removal agents, especially ethoxylated tetraethylene pentamine, dispersing agents, especially polyacrylates and polyasparatates, brighteners, especially anionic brighteners, suds suppressors, especially silicones and secondary alcohols, fabric softeners, especially smectite clays, and the like can all be used in such compositions at levels ranging from about 1% to about 35% by weight. Standard formularies and published patents contain multiple, detailed descriptions of such conventional materials.

Enzyme stabilizers may also be used in the cleaning compositions. Such enzyme stabilizers include propylene glycol (preferably from about 1% to about 10%), sodium form ate (preferably from about 0.1% to about 1%) and calcium formate (preferably from about 0.1% to about 1%).

The present variants are useful in hard surface cleaning compositions. As used herein “hard surface cleaning composition” refers to liquid and granular detergent compositions for cleaning hard surfaces such as floors, walls, bathroom tile, and the like. Hard surface cleaning compositions of the present invention comprise an effective amount of one or more variants of the present invention, preferably from about 0.001% to about 10%, more preferably from about 0.01% to about 5%, more preferably still from about 0.05% to about 1% by weight of variant of the composition. In addition to comprising one or more of the variants, such hard surface cleaning compositions typically comprise a surfactant and a water-soluble sequestering builder. In certain specialized products such as spray window cleaners, however, the surfactants are sometimes not used since they may produce a filmy and/or streaky residue on the glass surface.

The surfactant component, when present, may comprise as little as 0.1% of the compositions herein, but typically the compositions will contain from about 0.25% to about 10%, more preferably from about 1% to about 5% of surfactant.

Typically the compositions will contain from about 0.5% to about 50% of a detergency builder, preferably from about 1% to about 10%.

Preferably the pH should be in the range of from about 7 to about 12. Conventional pH adjustment agents such as sodium hydroxide, sodium carbonate or hydrochloric acid can be used if adjustment is necessary.

Solvents may be included in the compositions. Useful solvents include, but are not limited to, glycol ethers such as diethyleneglycol monohexyl ether, diethyleneglycol monobutyl ether, ethyleneglycol monobutyl ether, ethyleneglycol monohexyl ether, propyleneglycol monobutyl ether, dipropyleneglycol monobutyl ether, and diols such as 2,2,4-trimethyl-1,3-pentanediol and 2-ethyl-1,3-hexanediol. When used, such solvents are typically present at levels of from about 0.5% to about 15%, more preferably from about 3% to about 11%.

Additionally, highly volatile solvents such as iso-propanol or ethanol can be used in the present compositions to facilitate faster evaporation of the composition from surfaces when the surface is not rinsed after “full strength” application of the composition to the surface. When used, volatile solvents are typically present at levels of from about 2% to about 12% in the compositions.

Hard surface cleaning compositions of the present invention are illustrated by the following examples.

### EXAMPLES 1-6

Liquid Hard Surface Cleaning Compositions

| | Ex. 1 | Ex. 2 | Ex. 3 | Ex. 4 | Ex. 5 | Ex. 6 |
|---|---|---|---|---|---|---|
| G70D-* + S78D-* + I79D-* | 0.05% | 0.50% | 0.02% | 0.03% | 0.30% | 0.05% |
| EDTA | — | — | 2.90% | 2.90% | — | — |
| Sodium Citrate | — | — | — | — | 2.90% | 2.90% |
| NaC₁₂ Alkyl-benzene | 1.95% | — | 1.95% | — | 1.95% | — |
| sulfonate | | | | | | |
| NaC₁₂ Alkylsulfate | — | 2.20% | — | 2.20% | — | 2.20% |
| NaC₁₂ (ethoxy) sulfate | — | 2.20% | — | 2.20% | — | 2.20% |
| C₁₂ Dimethylamine oxide | — | 0.50% | — | 0.50% | — | 0.50% |
| Sodium cumene sulfonate | 1.30% | — | 1.30% | — | 1.30% | — |
| Hexyl Carbitol | 6.30% | 6.30% | 6.30% | 6.30% | 6.30% | 6.30% |
| Water | 90.4% | 88.3% | 87.53% | 85.87% | 87.25% | 85.85% |

All formulas are adjusted to pH 7.

In Examples 1-6, the variants recited in Tables 2-3, and the preferred variants cited herein, among others, are substituted for G70D-*+S78D-*+I79D-*, with substantially similar results.

In another embodiment of the present invention, dishwashing compositions comprise one or more variants of the present invention. As used herein, “dishwashing composition” refers to all forms of compositions for cleaning dishes including, but not limited to, granular and liquid forms. Dishwashing compositions of the present invention are illustrated by the following examples.

### EXAMPLES 7-10

Liquid Dish Detergent

| | | | |
|---|---|---|---|
| Ex. 7 | Ex. 8 | Ex. 9 | Ex. 10 |

| | | | | |
|---|---|---|---|---|
| G70D-* + L75D-* + | 0.05% | 0.50% | 0.02% | 0.40% |
| N76D-* + N77D-* + | | | | |
| S78D-* + I79D-* + | | | | |
| G80D-* + V81D-* + | | | | |
| L82D-* | | | | |
| C₁₂-C₁₄ N-methyl glucamide | 0.90% | 0.90% | 0.90% | 0.90% |
| C₁₂ ethoxy (1) sulfate | 12.0% | 12.0% | 12.0% | 12.0% |
| 2-Methyl undecanoic acid | 4.50% | 4.50% | 4.50% | 4.50% |
| C₁₂ ethoxy (2) carboxylate | 4.50% | 4.50% | 4.50% | 4.50% |
| C₁₂ alcohol ethoxylate (4) | 3.00% | 3.00% | 3.00% | 3.00% |
| C₁₂ amine oxide | 3.00% | 3.00% | 3.00% | 3.00% |
| Sodium cumene sulfonate | 2.00% | 2.00% | 2.00% | 2.00% |
| Ethanol | 4.00% | 4.00% | 4.00% | 4.00% |
| Mg²⁺ (as MgCl₂) | 0.20% | 0.20% | 0.20% | 0.20% |
| Ca²⁺ (as CaCl₂) | 0.40% | 0.40% | 0.40% | 0.40% |
| Water | 65.45% | 65% | 65.48% | 65.1% |

All formulas are adjusted to pH 7.

In Examples 7-10, the variants recited in Tables 2-3, and the preferred variants cited herein, among others, are substituted for G70D-*+L75D-*+N76D-*+N77D-*+S78D-*+I79D-*+G80D-*+V81D-*+L82D-*, with substantially similar results.

Liquid fabric cleaning compositions of the present invention are illustrated by the following examples.

### EXAMPLES 11-13

Liquid Fabric Cleaning Compositions

| | Ex. 11 | Ex. 12 | Ex. 13 |
|---|---|---|---|
| G70D-* | 0.05% | 0.03% | 0.30% |
| Sodiuam C₁₂-C₁₄ alkyl sulfate | 20.0% | 20.0% | 20.0% |
| 2-Butyl octanoic acid | 5.0% | 5.0% | 5.0% |
| Sodium citrate | 1.0% | 1.0% | 1.0% |
| C₁₀ Alcohol ethoxylate (3) | 13.0% | 13.0% | 13.0% |
| Monoethanolamine | 2.50% | 2.50% | 2.50% |
| Water/propylene glycol/ethanol | 58.45% | 58.47% | 58.20% |
| (100:1:1) | | | |

Personal Care Compositions

The present variants are particularly suited for use in personal care compositions such as, for example, leave-on and rinse-off hair conditioners, shampoos, leave-on and rinse-off acne compositions, facial milks and conditioners, shower gels, soaps, foaming and non-foaming facial cleansers, cosmetics, hand, facial, and body lotions and moisturizers, leave-on facial moisturizers, cosmetic and cleansing wipes, oral care compositions, and contact lens care compositions. The present personal care compositions comprise one or more variants of the present invention and a personal care carrier.

To illustrate, the present variants are suitable for inclusion in the compositions described in the following references: U.S. Pat. No. 5,641,479, Linares et al., issued Jun. 24, 1997 (skin cleansers); U.S. Pat. No. 5,599,549, Wivell et al., issued Feb. 4, 1997 (skin cleansers); U.S. Pat. No. 5,585,104, Ha et al., issued Dec. 17, 1996 (skin cleansers); U.S. Pat. No. 5,540,852, Kefauver et al., issued Jul. 30, 1996 (skin cleansers); U.S. Pat. No. 5,510,050, Dunbar et al., issued Apr. 23, 1996 (skin cleansers); U.S. Pat. No. 5,612,324, Guang Lin et al., issued Mar. 18, 1997 (anti-acne preparations); U.S. Pat. No. 5,587,176, Warren et al., issued Dec. 24, 1996 (anti-acne preparations); U.S. Pat. No. 5,549,888, Venkateswaran, issued Aug. 27, 1996 (anti-acne preparations); U.S. Pat. No. 5,470,884, Corless et al., issued Nov. 28, 1995 (anti-acne preparations); U.S. Pat. No. 5,650,384, Gordon et al., issued Jul. 22, 1997 (shower gels); U.S. Pat. No. 5,607,678, Moore et al., issued Mar. 4, 1997 (shower gels); U.S. Pat. No. 5,624,666, Coffindaffer et al., issued Apr. 29, 1997 (hair conditioners and/or shampoos); U.S. Pat. No. 5,618,524, Bolich et al., issued Apr. 8, 1997 (hair conditioners and/or shampoos); U.S. Pat. No. 5,612,301, Inman, issued Mar. 18, 1997 (hair conditioners and/or shampoos); U.S. Pat. No. 5,573,709, Wells, issued Nov. 12, 1996 (hair conditioners and/or shampoos); U.S. Pat. No. 5,482,703, Pings, issued Jan. 9, 1996 (hair conditioners and/or shampoos); U.S. Pat. No. Re. 34,584, Grote et al., Reissued Apr. 12, 1994 (hair conditioners and or shampoos); U.S. Pat. No. 5,641,493, Date et al., issued Jun. 24, 1997 (cosmetics); U.S. Pat. No. 5,605,894, Blank et al., issued Feb. 25, 1997 (cosmetics); U.S. Pat. No. 5,585,090, Yoshioka et al., issued Dec. 17, 1996 (cosmetics); U.S. Pat. No. 4,939,179, Cheney et al., issued Jul. 3, 1990 (hand, face, and/or body lotions); U.S. Pat. No. 5,607,980, McAtee et al., issued Mar. 4, 1997 (hand, face, and/or body lotions); U.S. Pat. No. 4,045,364, Richter et al., issued Aug. 30, 1977 (cosmetic and cleansing wipes); European Patent Application, EP 0 619 074, Touchet et al., published Oct. 12, 1994 (cosmetic and cleansing wipes); U.S. Pat. No. 4,975,217, Brown-Skrobot et al., issued Dec. 4, 1990 (cosmetic and cleansing wipes); U.S. Pat. No. 5,096,700, Seibel, issued Mar. 17, 1992 (oral cleaning compositions); U.S. Pat. No. 5,028,414, Sampathkumar, issued Jul. 2, 1991 (oral cleaning compositions); U.S. Pat. No. 5,028,415, Benedict et al., issued Jul. 2, 1991 (oral cleaning compositions); U.S. Pat. No. 5,028,415, Benedict et al., issued Jul. 2, 1991 (oral cleaning compositions); U.S. Pat. No. 4,863,627, Davies et al., Sep. 5, 1989 (contact lens cleaning solutions); U.S. Pat. No. Re. 32,672, Huth et al, reissued May 24, 1988 (contact lens cleaning solutions); and U.S. Pat. No. 4,609,493, Schafer, issued Sep. 2, 1986 (contact lens cleaning solutions).

To further illustrate oral cleaning compositions of the present invention, a pharmaceutically-acceptable amount of one or more variants of the present invention are included in compositions useful for removing proteinaceous stains from teeth or dentures. As used herein, “oral cleaning compositions” refers to dentifrices, toothpastes, toothgels, toothpowders, mouthwashes, mouth sprays, mouth gels, chewing gums, lozenges, sachets, tablets, biogels, prophylaxis pastes, dental treatment solutions, and the like. Preferably, the oral cleaning compositions comprise from about 0.0001% to about 20% of one or more variants of the present invention, more preferably from about 0.001% to about 10%, more preferably still from about 0.01% to about 5%, by weight of the composition, and a pharmaceutically-acceptable carrier. As used herein, “pharmaceutically-acceptable” means that drugs, medicaments or inert ingredients which the term describes are suitable for use in contact with the tissues of humans and lower animals without undue toxicity, incompatibility, instability, irritation, allergic response, and the like, commensurate with a reasonable benefit/risk ratio.

Typically, the pharmaceutically-acceptable oral cleaning carrier components of the oral cleaning components of the oral cleaning compositions will generally comprise from about 50% to about 99.99%, preferably from about 65% to about 99.99%, more preferably from about 65% to about 99%, by weight of the composition.

The pharmaceutically-acceptable carrier components and optional components which may be included in the oral cleaning compositions of the present invention are well known to those skilled in the art. A wide variety of composition types, carrier components and optional components useful in the oral cleaning compositions are disclosed in the references cited hereinabove.

In another embodiment of the present invention, denture cleaning compositions for cleaning dentures outside of the oral cavity comprise one or more variants of the present invention. Such denture cleaning compositions comprise an effective amount of one or more of the variants, preferably from about 0.0001% to about 50% of one or more of the variants, more preferably from about 0.001% to about 35%, more preferably still from about 0.01% to about 20%, by weight of the composition, and a denture cleansing carrier. Various denture cleansing composition formats such as effervescent tablets and the like are well known in the art (see, e.g., U.S. Pat. No. 5,055,305, Young), and are generally appropriate for incorporation of one or more of the variants for removing proteinaceous stains from dentures.

In another embodiment of the present invention, contact lens cleaning compositions comprise one or more variants of the present invention. Such contact lens cleaning compositions comprise an effective amount of one or more of the variants, preferably from about 0.01% to about 50% of one or more of the variants, more preferably from about 0.01% to about 20%, more preferably still from about 1% to about 5%, by weight of the composition, and a contact lens cleaning carrier. Various contact lens cleaning composition formats such as tablets, liquids and the like are well known in the art and are generally appropriate for incorporation of one or more variants of the present invention for removing proteinaceous stains from contact lenses.

The contact lens cleaning composition embodiment of the present invention is illustrated by Examples 14-17.

### EXAMPLES 14-17

Contact Lens Cleaning Solution

| | Ex. 14 | Ex. 15 | Ex. 16 | Ex. 17 |
|---|---|---|---|---|
| N76D-* | 0.01% | 0.5% | 0.1% | 2.0% |
| Glucose | 50.0% | 50.0% | 50.0% | 50.0% |
| Nonionic surfactant | 2.0% | 2.0% | 2.0% | 2.0% |
| (polyoxyethlene - pol | | | | |
| Anionic surfactant | 1.0% | 1.0% | 1.0% | 1.0% |
| (polyoxyethylene - | | | | |
| alkylphenylether sodium | | | | |
| sulfricester) | | | | |
| Sodium Chloride | 1.0% | 1.0% | 1.0% | 1.0% |
| Borax | 0.30% | 0.30% | 0.30% | 0.30% |
| Water | 45.69% | 45.20% | 45.60% | 43.70% |

Examples 18-21 illustrate the use of the present variants in bodywash products:

### EXAMPLES 18-21

Bodywash Products

| | Ex. 18 | Ex. 19 | Ex. 20 | Ex. 21 |
|---|---|---|---|---|
| Water | 62.62% | 65.72% | 57.72% | 60.72% |
| Disodium EDTA | 0.2% | 0.2% | 0.2% | 0.2% |
| Glycerine | 3.0% | 3.0% | 3.0% | 3.0% |
| Polyquaternium 10 | 0.4% | 0.4% | 0.4% | 0.4% |
| Sodium laureth sulphate | 12.0% | 12.0% | 12.0% | 12.0% |
| Cocamide MEA | 2.8% | 2.8% | 2.8% | 2.8% |
| Sodium lauraphoacetate | 6.0% | 6.0% | 6.0% | 6.0% |
| Myristic Acid | 1.6% | 1.6% | 1.6% | 1.6% |
| Magnesium sulphate heptahydrate | 0.3% | 0.3% | 0.3% | 0.3% |
| Trihydroxystearin | 0.5% | 0.5% | 0.5% | 0.5% |
| PEG-6 caprylic/capric triglycerides | 3.0% | — | — | — |
| Sucrose polyesters of cottonate fatty acid | 3.0% | — | — | — |
| Sucrose polyesters of behenate fatty acid | 3.0% | — | 4.0% | — |
| Petrolatum | — | 4.0% | 8.0% | — |
| Mineral Oil | — | — | — | 6.0% |
| DMDM Hydantoin | 0.08% | 0.08% | 0.08% | 0.08% |
| L75D-* + N76D-* + N77D-* + S78D-* + | 0.1% | 2.0% | 2.0% | 5.0% |
| 179D-* + G80D-* + V81D-* + L82D-* | | | | |
| Citric Acid | 1.40% | 1.40% | 1.40% | 1.40% |

In Examples 14-17, the variants recited in Tables 2-3, and the preferred variants cited herein, among others, are substituted for N76D-*, with substantially similar results.

Examples 22-25 illustrate the use of the present variants in facewash products:

### EXAMPLES 22-25

Facewash Products

| | Ex. 22 | Ex. 23 | Ex. 24 | Ex. 25 |
|---|---|---|---|---|
| Water | 66.52% | 65.17% | 68.47% | 68.72% |
| Disodium EDTA | 0.1% | 0.1% | 0.2% | 0.2% |
| Citric Acid | — | — | 1.4% | 1.4% |
| Sodium Laureth-3 Sulfate | 3.0% | 3.5% | — | — |
| Sodium Laureth-4 Carboxylate | 3.0% | 3.5% | — | — |
| Laureth-12 | 1.0% | 1.2% | — | — |
| Polyquaternium 10 | — | — | 0.4% | 0.4% |
| Polyquaternium 25 | 0.3% | 0.3% | — | — |
| Glycerine | 3.0% | 3.0% | 3.0% | 3.0% |
| Sodium Lauroamphoacetate | — | — | 6.0% | 6.0% |
| Lauric Acid | 6.0% | 6.0% | 3.0% | 3.0% |
| Myristic Acid | — | — | 3.0% | 3.0% |
| Magnesium sulphate heptahydrate | 2.3% | 2.0% | 2.0% | 2.0% |
| Triethanol amine | 4.0% | 4.0% | 4.0% | 4.0% |
| Trihydroxystearin | 0.5% | 0.5% | 0.5% | 0.5% |
| Sucrose polyesters of behenate fatty acid | 2.0% | 2.0% | — | — |
| Sucrose polyesters of cottonate fatty acid | 3.0% | 2.0% | — | — |
| PEG-6 caprylic/capric triglycerides | — | — | — | 2.0% |
| Petrolatum | — | — | 4.0% | — |
| Mineral Oil | — | — | — | 2.0% |
| Cocamidopropyl betaine | 2.0% | 3.0% | 1.8% | 1.8% |
| Lauryl dimethylamine oxide | 1.0% | 1.2% | 1.2% | 1.2% |
| Dex Panthenol | 1.0% | 0.25% | 0.25% | — |
| DMDM Hydantoin | 0.08% | 0.08% | 0.08% | 0.08% |
| L75D-* | 1.0% | 2.0% | 0.5% | 0.5% |
| Fragrance | 0.2% | 0.2% | 0.2% | 0.2% |

Examples 26-27 illustrate the use of the present variants in leave-on skin moisturizing compositions:

### EXAMPLES 26-27

Leave-on Skin Moisturizing Composition

| | Ex. 26 | Ex. 27 |
|---|---|---|
| Glycerine | 5.0% | — |
| Stearic acid | 3.0% | — |
| C₁₁₋₁₃ Isoparaffin | 2.0% | — |
| Glycol stearate | 1.5% | — |
| Propylene glycol | — | 3.0% |
| Mineral oil | 1.0% | 10.0% |
| Sesame oil | — | 7.0% |
| Petrolatum | — | 1.8% |
| Triethanolamine | 0.7% | — |
| Cetyl acetate | 0.65% | — |
| Glyceryl stearate | 0.48% | 2.0% |
| TEA stearate | — | 2.5% |
| Cetyl alcohol | 0.47% | — |
| Lanolin alcohol | — | 1.8% |
| DEA —cetyl phosphate | 0.25% | — |
| Methylparaben | 0.2% | 0.2% |
| Propylparaben | 0.12% | 0.1% |
| Carbomer 934 | 0.11% | — |
| Disodium EDTA | 0.1% | — |
| L82D-* | 0.1% | 0.5% |
| Water | 84.32% | 71.1% |

In Examples 26-27, the variants recited in Tables 2-3, and the preferred variants cited herein, among others, are substituted for L82D-*, with substantially similar results.

Example 28 illustrates the use of the present variants in cleansing wipe compositions:

### EXAMPLE 28

Cleansing Wipe Composition

| | |
|---|---|
| Propylene Glycol | 1.0% |
| Ammonium lauryl sulfate | 0.6% |
| Succinic acid | 4.0% |
| Sodium succinate | 3.2% |
| Triclosan ® | 0.15% |
| S78D-* | 0.05% |
| Water | 91.0% |

The above composition is impregnated onto a woven absorbent sheet comprised of cellulose and/or polyester at about 250%, by weight of the absorbent sheet.

In Example 28, the variants recited in Tables 2-3, and the preferred variants cited herein, among others, are substituted for S78D-*, with substantially similar results.

What is claimed is:

1. An enzyme variant having a modified amino acid sequence of a wild-type amino acid sequence comprising a substitution by a substituting D-amino acid at one or more amino acid positions, wherein at least one amino acid of the enzyme variant is an L-amino acid.

2. An enzyme variant according to claim 1 wherein at least one of the substitutions occurs in an epitope region of the wild-type amino acid sequence.

3. An enzyme variant according to claim 2 wherein the epitope region is a T-cell epitope region.

4. An enzyme variant according to claim 3 which is a variant of an enzyme selected from the group consisting of proteases, cellulases, lipases, amylases, peroxidases, microperoxidases, hemicellulases, xylanases, phospholipases, esterases, cutinases, pectinases, keratinases, reductases, oxidases, phenoloxidases, lipoxygenases, ligninases, pullulanases, tannases, pentosanases, malanases, b-glucanases, arabinosidases, hyaluronidases, chondroitinases, laccases, transferases, isomerases, lyases, ligases, synthetases, and fruit-based enzymes.

5. An enzyme variant according to claim 4 wherein the enzyme is a protease.

6. An enzyme variant according to claim 5 wherein the protease is a serine protease.

7. An enzyme variant according to claim 6 wherein the wild-type amino acid sequence comprises a first epitope region, a second epitope region, and a third epitope region, wherein the modified amino acid sequence comprises a substitution by a substituting D-amino acid at one or more positions in one or more of the epitope regions wherein:
(i) when a substitution occurs in the first epitope region, the substitution occurs at one or more positions corresponding to positions 70-84 of subtilisin BPN′;
(ii) when a substitution occurs in the second epitope region, the substitution occurs at one or more positions corresponding to positions 103-126 of subtilisin BPN′; and
(iii) when a substitution occurs in the third epitope region, the substitution occurs at one or more positions corresponding to positions 217-252 of subtilisin BPN′.

8. An enzyme variant according to claim 7 wherein the serine protease is selected from the group consisting of subtilisin BPN′, subtilisin Carlsberg, subtilisin DY, subtilisin 309, proteinase K, and thermitase.

9. An enzyme variant according to claim 8 wherein the serine protease is subtilisin BPN′.

10. An enzyme variant according to claim 9 wherein at least one substitution occurs in the first epitope region.

11. An enzyme variant according to claim 10 wherein at least one substitution is of one or more of positions 70 and 75-82 corresponding to subtilisin BPN′.

12. An enzyme variant according to claim 10 wherein at least one substitution occurs in the second epitope region.

13. A protein variant according to claim 10 wherein at least one substitution occurs in the third epitope region.

14. A protein variant according to claim 10 further comprising one or more stabilizing mutations, wherein the stabilizing mutations are each, independently, by a substituting L-amino acid or D-amino acid.

15. A cleaning composition comprising an enzyme variant according to claim 1 and a cleaning composition carrier.

16. A personal care composition comprising an enzyme variant according to claim 1 and a personal care carrier.

17. A mutant gene encoding the enzyme variant according to claim 1.
